(19) United States
(12) Patent Application Publication
(10) Pub. No.: US 2007/0017535 A1
(43) Pub. Date: Jan. 25, 2007

(54) System for tracking surgical instruments

(75) Inventors: Milton Frank, Bergenfield, NJ (US); Frank Froelich, Bergenfield, NJ (US)

Correspondence Address: EPSTEIN DRANGEL BAZERMAN & JAMES, LLP, 60 EAST 42ND STREET, SUITE 820, NEW YORK, NY, 10165, US

(73) Assignee: Med-Track Partners, LLC

(21) Appl. No.: 11/489,340

(22) Filed: Jul. 19, 2006

Related U.S. Application Data
(60) Provisional application No. 60/701,356, filed on Jul. 21, 2005.

(51) Int. Cl. A61B 19/00

(52) U.S. Cl. 128/898

(57) ABSTRACT

The present invention relates to a computer-implemented method of tracking surgical instruments which begins with creating a virtual surgical kit. The number of each instrument selected for use in the surgery is stored to form the pre-surgical count for the virtual kit. The number of each instrument actually present during or after surgery is counted and is entered into the computer. The computer compares the pre-surgical count with the entered count and displays any difference between those counts, indicating that a surgical instrument is missing.

### BACKGROUND OF THE INVENTION

1. Field of the Invention

The present invention relates to a system for tracking surgical instruments and, in particular, a computer implemented system for tracking surgical instruments that increases the accuracy of surgical instrument and sponge counting so as to reduce the incidence of instruments and sponges being accidentally left in the body of surgical patients after surgery.

2. Description of Prior Art including Information Disclosed under 37 CFR 1.97 and 1.98

A surgical procedure may require the use of many surgical instruments and sponges. Failure to remove all instruments and sponges from the body of the surgical patient after a procedure may cause serious complications and require further surgery.

Further, a retained instrument or sponge may not cause an immediate reaction. Rather, the symptoms of the complication may not be noticeable until some time after the surgery. This might lead to doctor and/or hospital visits to diagnose the problem, without the knowledge of the original surgery.

The current method of tracking the surgical instruments and sponges relies on count sheets. All instruments required to be placed into a particular surgical set are listed on these count sheets. There is generally a method for noting the instrument and its count as it is placed in the set. This paper count sheet must then accompany the surgical set through sterilization and on to the operating room.

Before the surgical procedure, the instruments are recounted as they are laid out and each instrument's count is again noted on the count sheet. The instrument count may also be repeated one or more times during the procedure and just before the procedure is ended. Sponges are handled on separate count sheets since they are introduced during the procedure as needed.

The above method of tracking surgical instruments and sponges has been shown to be inaccurate. The count taken before the surgical procedure is sometimes different from that on the count sheet. This requires a delay in the surgical procedure until replacement sterilized instruments are brought to the operating room.

Further, instrument counts taken by the same person during the surgical procedure may be different. On the other hand, instruments and sponges that were correctly counted after the surgical procedure have been found later to have been retained by the patient.

The paper count sheets are easily misplaced or soiled. That leaves no permanent record of that procedure's instrument count.

The tendency for humans to see what they expect must also be taken into account. With the original instrument counted listed on the sheet, anyone recounting knows what the count should be and may mistakenly enter the expected count on the sheet, rather than the actual count.

### BRIEF SUMMARY OF THE INVENTION

Accordingly, it is a prime object of the present invention to provide a computer-implemented system of tracking surgical instruments that increases the accuracy of surgical instrument and sponge counting, and therefore lowers the incidence of retained instruments or sponges.

For purposes of simplicity, the system of the present invention is described and claimed herein using the term “instrument” to refer to the objects being tracked. However, it should be understood that “instrument” as that term is used herein is intended broadly to include any object, including a sponge, that may be used as part of a surgical procedure. In addition, the word “surgery” is used generically to mean any medical or surgical procedure in which instruments are used.

In accordance with one aspect of the present invention, a computer-implemented method of tracking surgical instruments is provided. The method begins by creating a virtual surgical instrument kit in the computer prior to surgery by selecting a name for the kit and associating a memory location with the kit. Each instrument to be used in surgery is designated to be added to the kit. The number of designated instruments to be used in surgery is selected to be added to the kit to form the pre-surgical count for the kit. The pre-surgical count of the kit is then stored in the computer.

During or after surgery, the stored kit is selected. The actual number of each of the designated instruments present at that time is counted. That number is entered into the computer to form the entered count of the stored kit. The computer then compares the pre-surgical count and the entered count of the selected kit and displays any difference between the pre-surgical count and the entered count of the stored kit.

The method further includes the step of creating an instruments database containing a list of surgical instruments. The step of creating an instruments database further includes storing an image of each instrument in the database.

The step of designating an instrument to be added to the kit may include accessing the instrument database; displaying the image of each instrument in the instrument database; selecting a displayed image; and adding the number of instruments depicted in the selected image to the pre-surgical count of the kit.

The step of designating an instrument to be added to the kit may also include designating an instrument to be added to the kit; selecting the number of the instrument designated to be added to the pre-surgical count; and adding the selected number into the pre-surgical count for the kit.

The method further includes the step of deleting a selected number of instruments present in the stored kit.

The method also includes displaying the contents of the stored kit and displaying the history of the stored kit.

The method also includes the step of protecting the instruments database by requiring a password for access thereto.

The method also includes the steps of entering hospital information into the database; creating a system log; viewing the system log and displaying an error in the system log.

The method further includes creating a data log; viewing the data log; and displaying an error in the data log.

The step of creating an instrument database includes obtaining information as to an instrument and adding same to the instrument database. It also includes selecting an instrument in the instruments database and deleting the selected instrument.

In accordance with another aspect of the present invention, a computer-implemented method of tracking surgical instruments is provided. The method begins with creating a virtual surgical kit. The number of each instrument selected for use in the surgery is stored to form the pre-surgical count for the virtual kit. The actual number of each instrument present during or after surgery is counted and entered into the computer as the entered count. The computer compares the pre-surgical count with the entered count and displays any difference between the pre-surgical count and the entered count.

The method further includes creating an instruments database containing a list of surgical instruments. That includes storing an image of each instrument in the database.

The step of storing the number of each selected instrument may include accessing the instrument database; displaying the image of each instrument in the instrument database; selecting a displayed image; and adding the number of instruments depicted in the selected image to the pre-surgical count of the kit.

The step of storing the number of each selected instrument may also include designating an instrument to be added to the kit; selecting the number of the designated instrument to be added to the count; and adding the selected number into the pre-surgical count for the kit.

The method also includes deleting a selected number of an instrument from the pre-surgical count.

The method further includes displaying the pre-surgical count for the kit and displaying the history of the kit.

The method also includes protecting the instruments database by requiring a password for access.

The step of creating an instrument database further includes obtaining information as to an instrument and adding same to the instrument database. It also includes selecting an instrument in the database and deleting the selected instrument.

### BRIEF DESCRIPTION OF THE SEVERAL VIEWS OF DRAWINGS

To these and to such other objects that may hereinafter appears, the present invention relates to a system of tracking surgical instruments as described in detail in the following specification and recited in the annexed claims, taken together with the accompanying drawings, in which like numerals refer to like parts and in which:

FIG. 1 is a representation of the main computer menu utilized in the performance of the method of the present invention; and

FIG. 2 is a representation of the configuration menu utilized in the performance of the method of the present invention.

### DETAILED DESCRIPTION OF THE INVENTION

This system of the present invention utilizes one or more computers, either stand alone or networked, running software that implements the present invention. The system maintains a computer database of surgical instruments as well as a database of surgical set contents. These databases may be stored on an individual computer or on the network.

Preferably, the surgical instrument database is pre-populated from a list of known surgical instruments. Surgical instruments may also be added to the database through data entry screens on the computer.

The surgical set database may be pre-populated from a list of known surgical sets. Surgical sets may also be added to the database through data entry screens on the computer.

Data may be entered into the computer by using a standard QWERTY style keyboard and a mouse device. The mouse and keyboard may also be used to select data on the screen for processing.

The use of a computer display screen with an integrated touchscreen is preferred for data entry and selection. Entry of the actual instrument count can be accomplished by utilizing a keyboard/mouse combination, an integrated touchscreen, or both. Entering text on a touchscreen computer that does not have a keyboard is accomplished via a QWERTY style keyboard displayed on the computer's display screen.

The computer may consist of separate components such as a display, a processing unit, a keyboard, and a mouse. However, an integrated computer where a single component contains the display, processing unit, and touchscreen is preferred.

For purposes of illustration, the method of the present invention described herein as utilizing a computer consisting of separate components, an integrated computer with touchscreen, or a combination of both. It should be understood, however, that other types of equipment could be used to implement the invention.

The present invention utilizes a computer system that maintains a log of all counts taken on each surgical set. For each count, the log contains the name of the surgical set, the names of the individuals doing the counting, and the expected (pre-surgical) and actual (taken during or after surgery) count for each instrument in the set.

When the actual count is taken during a procedure, the log also contains the operating room name or number, the patient's name, and the expected and actual count of any sponges added during the procedure. At the end of a procedure, the log of that procedure may be printed and placed with the patient's record. The complete log of any surgical set may be displayed or printed if needed. Any incorrect instrument or sponge counts are also logged in an error log that keeps a permanent record of any errors. This error log may be viewed or printed as needed.

The present invention utilizes a method of counting surgical instruments and sponges that displays the instruments and sponges on the computer display screen along with an area to enter the count for each instrument and sponge. The individual doing the countering determines the number of each instrument or sponge and enters that number into the computer via the touchscreen or keyboard. At the completion of the count, the actual count is compared against the expected (pre-surgical) count and the results are displayed on the computer screen, as well as being added to the surgical kit's permanent log.

Further, the present invention anticipates that the surgical set will be recounted before sterilization to maintain the accuracy of the instrument count for that surgical set.

Following are a list of the requirements of the computer system necessary to implement the method of the present invention:

| | |
|---|---|
| Processor: | Intel Pentium/Celeron, AMD K6/Athlon/Duron, Via Eden, or |
| | compatible processor. |
| Processor | 300 megahertz or greater |
| Speed: | |
| Processor | 256 megabytes |
| Memory: | |
| Storage: | 20 gigabyte hard disk. |
| | CD-ROM for software installation |
| Operating | Microsoft Windows ® 2000 or Microsoft Windows ® XP |
| System: | |
| Video | Capable of a resolution of 1024 × 768 and 32 bit true color. |
| Adapter: | |
| Monitor: | 15″ Diagonal capable of displaying 1024 × 768 resolution and |
| | 32 bit color. The software is designed to work with a touch |
| | screen monitor and the touch screen is highly recommended. |
| Keyboard: | Only required if not using a touch monitor. |
| Mouse: | Only required if not using a touch monitor. |
| USB port: | Not required for this implementation but may be required by |
| | the touch monitor. |
| RS-232 | One required for the optional printer. A second port may be |
| Serial | required if using a touch monitor. |
| port: | |
| Network | 101100 Base-T or 802.1 lb required if multiple computers are |
| Adapter: | sharing the same database. |

The software used to implement the method of the present invention preferably includes a pre-programmed instrument database.

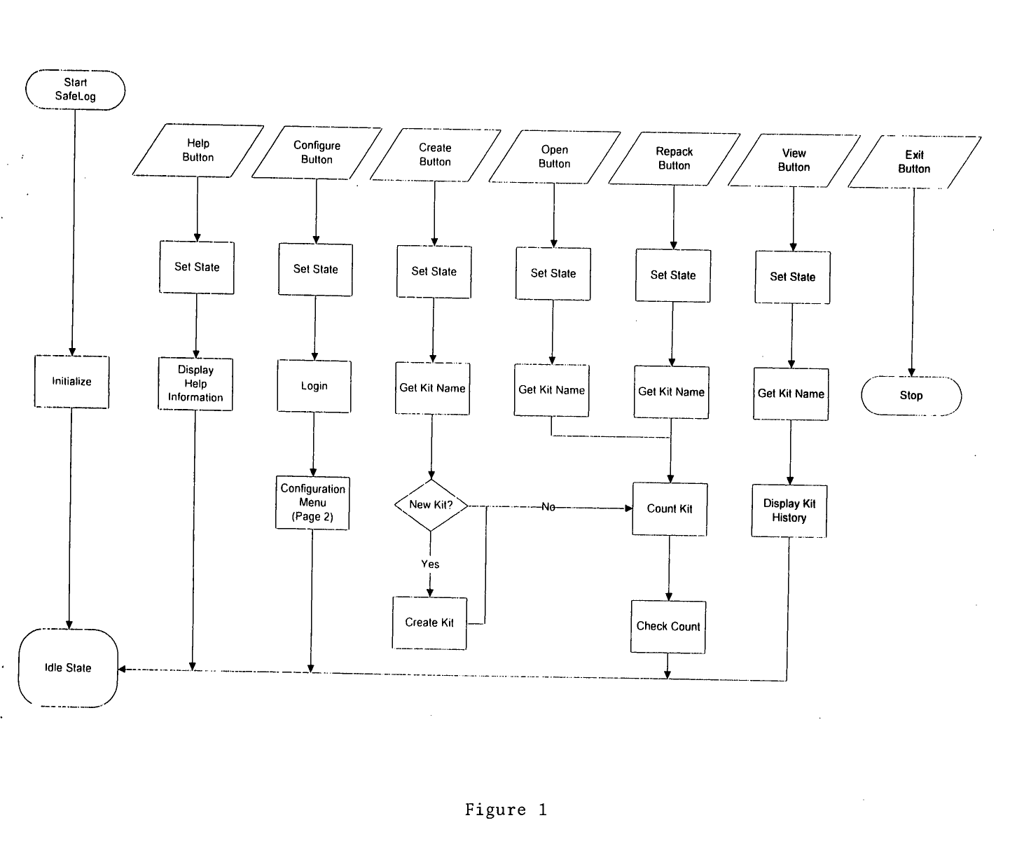

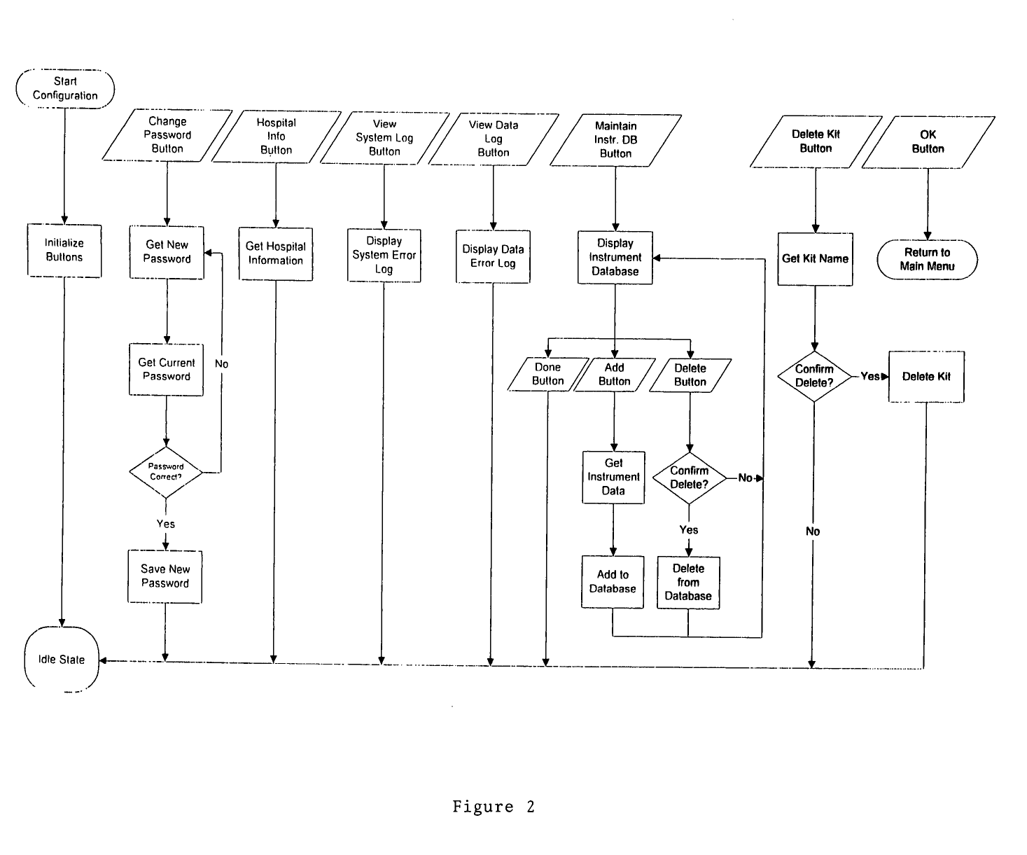

Following is a description of the function of each of the buttons available in the Main Menu that appears on the computer screen during the implementation of the present invention, as depicted in FIG. 1:
Help button—provides the user with a display of information about the operation of the software used to implement the present invention.
Configuration & Maintenance button—allows entry to Configuration Menu, FIG. 2, using the user password.
Create button—permits user to create a new kit and enter the pre-surgical count of instruments into the new kit.
Open button—permits user to open an existing kit for use. User will be asked to select a kit and then enter his or her user information. The screen will display the contents of the selected kit.
Repack button—permits user to enter an instrument count into selected kit after use and to check count against kit history.
View or print button—allows user to display or print the count history of a selected kit.
Exit button—Shuts down the application.

When a newly created or repacked kit is displayed, the screen displays two windows. One window displays a list of instruments (preferably in the form of the image of each instrument) from the instrument database. The other window displays the contents of the selected kit.

In order to add a particular instrument to the selected kit from the instrument database, the user double clicks on the instrument name or displayed instrument image and that instrument is added to the kit contents. Double clicking on an instrument that is already in the kit will increase the count of that instrument in the kit. Double clicking on the image of an item listed in the kit display will remove it from the kit.

An “Add items” window and a button are available to enter into or remove instruments from the current kit that are not present in the instruments database. The first time an instrument is added through the “Add Items” window, it will be added to the list in the instrument database.

There also is a search window and button. Entering part of the name of an instrument and pressing the search button will search the database memory and find the first line that matches the text and move it into the visible part of the tools window. The toolbar buttons are as follows:
Save button—saves the kit contents under a user selected name.
Back button—go back to the main menu.

Pressing the Open button causes the display of a window that shows the existing kits and asks the user to select one. The names of the individuals doing the counting, the operating room, and the name of the patient are then requested.

The contents of the selected kit are displayed. An “Add items” window and buttons are available to enter or remove new items, especially sponges, into the kit. The toolbar buttons are as follows:
Count button—counts the instruments in the kit.
Back button—go back to the main menu.

Pressing the Count button causes the contents of the selected kit to be displayed along with a numeric keypad. An instrument is selected from the database, or using the Add items button, as explained above, and the count of that instrument is entered using the keypad. The up & down arrows on the keypad will move through the instruments displayed in the window. The toolbar buttons are as follows:
Check button—compares the entered (post-surgical) counts with the expected (pre-surgical) counts displaying both counts on the screen with any errors in red.
Back button—back to the Open menu.

The Check Count button is used to compare the entered (post-surgical) count to the expected (pre-surgical) count and displays any errors in red.

The toolbar buttons are:
Count button—causes a recount of the instruments in the kit. Only displayed if there are errors.
Print button—causes the checked results to be printed.
Back—go back to the main menu.

Pressing the View button causes the display of a window which shows the existing kits and asking the user to select one. The usage history of the selected kit is then displayed. Any count errors are noted by asterisks.

The toolbar buttons are as follows:
Print button—causes the usage history to be printed.
Back button—back to the main menu.

The Configuration and Maintenance button permits the user to enter the password-protected Configuration Menu of the present invention, illustrated on FIG. 2. That menu offers several buttons for configuring and maintaining the system.

Following is a description of the function of each of the buttons available in the Configuration Menu screen, as depicted in FIG. 2:
Change Password button—This section requires the current password, a new password and confirmation of the new password.
Enter Hospital Information button—This allows the user to enter the hospital's name, address, city, state, ZIP, and phone number.
View System Error Log button—allows the user to view or print the log of errors that are system related.
View Data Error Log button—allows the user to view or print the log of errors that are directly related to instrument counting.
Maintain Instrument List button—allows the user to add or delete instruments from the instrument database.
Delete kit button—allows the user to delete surgical kits from the kit database.
Select Printer button—This button allows the user to select: None, a pre-programmed printer selection, or Select a printer from a list. Select from a list displays the standard printer dialog before printing.

It will now be appreciated that the present invention relates to a computer-implemented method of tracking surgical instruments which begins with creating a virtual surgical kit. The number of each instrument selected for use in the surgery is stored to form the entered pre-surgical count for the virtual kit. The number of each instrument actually present during or after surgery is counted and is entered into the computer. The computer compares the pre-surgical count with the entered count and displays any difference between those counts, indicating that a surgical instrument is missing.

While only a single preferred embodiment of the present invention has been disclosed for purposes of illustration, it is obvious that many modifications and variations could be made thereto. It is intended to cover all of those modifications and variations which fall within the scope of the present invention, as defined by the following claims.

We claim:

1. A computer-implemented method of tracking surgical instruments comprising the steps of:
(a) creating a virtual surgical instrument kit prior to surgery by selecting a name for the kit and associating a memory location with the kit;
(b) designating an instrument to be used in surgery to be added to the kit;
(c) selecting the number of designated instruments to be used in surgery to be added to the kit to form the pre-surgical count; and
(d) storing the pre-surgical count of the kit.

2. The method of claim 1 further comprising the steps of:
(a) selecting the stored kit;
(b) counting the number of each of the designated instruments actually present;
(c) entering the counted number to form the entered count of the stored kit;
(d) causing the computer to compare the pre-surgical count and the entered count of the selected kit; and
(e) displaying any difference between the pre-surgical count and entered count of the stored kit.

3. The method of claim 1 further comprising the step of creating an instruments database containing a list of surgical instruments.

4. The method of claim 3 wherein the step of creating an instruments database further comprises storing an image of each instrument in the database.

5. The method of claim 4 wherein the step of designating an instrument to be added to the kit comprises the steps of:
(a) accessing the instrument database;
(b) displaying the image of each instrument in the instrument database;
(c) selecting a displayed image; and
(d) adding the number of the instruments depicted in the selected image to the pre-surgical count of the kit.

6. The method of claim 3 wherein the step of designating an instrument to be added to the kit comprises the step of:
(a) designating an instrument to be added to the kit;
(b) selecting the number of the instrument designated to be added to the pre-surgical count; and
(c) adding the selected number into the pre-surgical count for the kit.

7. The method of claim 1 further comprising the step of deleting a selected number of instruments present in the stored kit.

8. The method of claim 1 further comprising the step of displaying the contents of the stored kit.

9. The method of claim 1 further comprising the step of displaying the history of the stored kit.

10. The method of claim 1 further comprising the step of protecting the instruments database by requiring a password for access thereto.

11. The method of claim 1 further comprising the step of entering hospital information into the database.

12. The method of claim 1 further comprising the step of creating a system log.

13. The method of claim 12 further comprising the step of viewing the system log.

14. The method of claim 12 further comprising the step of displaying an error in the system log.

15. The method of claim 1 further comprising the step of creating a data log.

16. The method of claim 12 further comprising the step of viewing the data log.

17. The method of claim 15 further comprising the step of displaying an error in the data log.

18. The method of claim 3 wherein the step of creating an instrument database further comprises the step of obtaining information as to an instrument and adding same to the instrument database.

19. The method of claim 3 further wherein the step of creating an instrument database further comprises the step of selecting an instrument in the instruments database and deleting the selected instrument.

20. A computer-implemented method of tracking surgical instruments comprising the steps of:
(a) creating a virtual surgical kit;
(b) storing the number of each instrument selected for use in the surgery to form the pre-surgical count;
(c) counting the number of each instrument present;
(d) entering the countered number to form the entered count;
(e) comparing the pre-surgical count with the entered count; and
(f) displaying any difference between the pre-surgical count and the entered count.

21. The method of claim 20 further comprising the step of creating an instruments database containing a list of surgical instruments.

22. The method of claim 21 wherein the step of creating an instruments database further comprises storing an image of each instrument in the database.

23. The method of claim 22 wherein the step of storing the number of each selected instrument comprises the steps of:
(a) accessing the instrument database;
(b) displaying the image of each instrument in the instrument database;
(c) selecting a displayed image; and
(d) adding the number of instruments depicted in the selected image to the pre-surgical count of the kit.

23. The method of claim 22 wherein the step of storing the number of each selected instrument comprises the steps of:
(a) designating an instrument to be added to the kit;
(b) selecting the number of the designated instrument to be added to the count; and
(c) adding the selected number into the pre-surgical count for the kit.

24. The method of claim 20 further comprising the step of deleting a selected number of an instrument from the pre-surgical count.

25. The method of claim 20 further comprising the step of displaying the pre-surgical count for the kit.

26. The method of claim 20 further comprising the step of displaying the history of the kit.

27. The method of claim 21 further comprising the step of protecting the instruments database by requiring a password for access.

28. The method of claim 21 further wherein the step of creating an instrument database further comprises the step of obtaining information as to an instrument and adding same to the instrument database.

29. The method of claim 21 further wherein the step of creating an instrument database further comprises the step of selecting an instrument in the database and deleting the selected instrument.
